(19) United States
(12) Patent Application Publication
(10) Pub. No.: US 2025/0041570 A1
(43) Pub. Date: Feb. 6, 2025

(54) IMPROVED DIAGNOSIS AND TREATMENT SELECTION THROUGH TISSUE PROPERTY DETERMINATION USING BALLOONING, IMAGING AND DIGITAL TWINS

(71) Applicant: KONINKLIJKE PHILIPS N.V., EINDHOVEN (NL)

(72) Inventors: Rene Leonardus Jacobus Marie UBACHS, EINDHOVEN (NL); Vipul Shrihari PAI RAIKAR, SOMERVILLE, MA (US)

(21) Appl. No.: 18/718,077

(22) PCT Filed: Dec. 5, 2022
(86) PCT No.: PCT/EP2022/084329

§ 371 Date: Jun. 10, 2024

Related U.S. Application Data
(60) Provisional application No. 63/290,340, filed on Dec. 16, 2021.

(51) Int. Cl. A61M 25/10; G16H 50/50; G16H 50/70

(52) U.S. Cl. CPC A61M 25/104; G16H 50/50; G16H 50/70

(57) ABSTRACT

A vascular therapy apparatus (1) includes an electronic processing device (18) including at least one electronic processor (20) programmed to while an angioplasty procedure is performed on a treatment site in a vessel (V) of a patient, obtain data about the angioplasty procedure; determine treatment information for a subsequent vascular therapy treatment of the treatment site by analyzing the data about the angioplasty procedure; and output the treatment information.

### FIELD

The following relates generally to the catheter arts, intravascular therapy arts, imaging arts, clot retrieval arts, intravascular therapy planning and monitoring arts, and related arts.

### BACKGROUND

A stent is a medical device often used in vascular therapy procedures (e.g., thrombectomy, atherectomy, and so forth) and are designed to remove or modify tissue or material from inside a diseased vessel (e.g., an artery, a vein, etc.). Different stents have been observed to have different radial outward force and stiffness (i.e., compliance). These factors, together with the amount of oversizing, determine the amount of patency reached after deployment and affect possible migration of the stent. In addition, tissue properties are important here, since the combination of stent and vessel, and surrounding tissue, determine the final lumen geometry. Tissue properties of the occlusion being treated and of the surrounding vascular tissue are different for each case and are typically not known or measured in vivo. Even in a single patient the properties will vary at different locations in the vasculature, e.g. due to local calcification.

Computations are typically performed to estimate the final geometry of a stented vein, after which the blood flow through the new lumen has been simulated. The flow has a large role in the amount of thrombus which can develop and hence on the restenosis of a treated lesion. The flow in its turn is heavily affected by the shape of the lumen and therefore it is beneficial to determine this accurately when performing outcome prediction. Hence, having better information on the properties of the vessel and surrounding tissue would be valuable.

The following discloses certain improvements to overcome these problems and others.

### SUMMARY

In some embodiments disclosed herein, a vascular therapy apparatus includes an electronic processing device comprising at least one electronic processor programmed to while an angioplasty procedure is performed on a treatment site in a vessel of a patient, obtain data about the angioplasty procedure; determine treatment information for a subsequent vascular therapy treatment of the treatment site by analyzing the data about the angioplasty procedure; and output the treatment information.

In some embodiments disclosed herein, a vascular therapy method includes performing an angioplasty procedure by expanding a balloon at a treatment site in a vessel of a patient; while performing the angioplasty procedure, obtaining data about the angioplasty procedure; analyzing the data about the angioplasty procedure using an electronic processor to determine treatment information for a subsequent vascular therapy treatment of the treatment site; and performing the subsequent vascular therapy treatment of the treatment site in accordance with the determined treatment information.

In some embodiments disclosed herein, a balloon angioplasty device includes a catheter; an expandable balloon disposed at a distal end of the catheter; and a sensor disposed inside the balloon and configured to measure data about an angioplasty procedure performed using the catheter.

One advantage resides in determining an appropriate stent size for a vascular therapy procedure.

Another advantage resides in determining an appropriate stent size for a vascular therapy procedure based on a tissue compliance of tissue at a treatment site of a patient.

Another advantage resides in determining an appropriate stent size for a vascular therapy procedure using a simulation model of tissue of a patient.

Another advantage resides in using imaging and/or sensor data to determine an appropriate stent size for a vascular therapy procedure.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a vascular therapy apparatus in accordance with the present disclosure.

FIG. 2 diagrammatically illustrates a method of performing a vascular therapy method using the apparatus of FIG. 1.

FIGS. 3A-B diagrammatically illustrate operations of the method of FIG. 2.

### DETAILED DESCRIPTION

The following relates to improved stent placement in a workflow in which a balloon angioplasty is first performed, followed by deploying the stent (or performing thrombectomy or other additional therapy) at the same location. For example, while balloon angioplasty procedures can be used to increase lumen of occluded vessels, often a decision needs to be taken whether a stent should subsequently be placed and, if so, what type and size of stent is optimal. These decisions are based on the limited data available and are heavily influenced by the knowledge and experience of the treating physician.

In an existing procedure of this kind (and using follow-up stent placement as an example), the surgeon first inserts a balloon angioplasty catheter to place the balloon at the target site, and then inflates the balloon to perform balloon angioplasty to deform the blockage so as to widen the vessel lumen. This may be done with real-time imaging (e.g. ultrasound (US) or computed tomography (CT)), so that the initial and widened vessel lumen diameters are thus determined. The balloon angioplasty catheter is then withdrawn, and a stent deployment catheter is inserted, and the stent is placed. In selecting the type of stent (stiffness, diameter, etc.), the surgeon relies primarily on experience/knowledge.

The following discloses an improved approach in which treatment information for a subsequent vascular therapy treatment of the treatment site (e.g., stenting) is determined by analyzing the data about the angioplasty procedure. In some embodiments, this is done using a digital twin (i.e. a computational simulation model representing the device and/or the patient which can be used to predict the outcome of a procedure. The accuracy of the prediction is determined in part by the quality and quantity of input data. Ideally, the virtual, in silico, behavior of the digital twin is an exact copy of the behavior of the physical, in vivo, behavior.) of a stenosis in conjunction with information obtained before, during, and/or after the angioplasty procedure to provide more information about the stenosis from which the stent or other follow-up therapy can be better selected. The digital twin models tissue properties (i.e., tissue compliance) of the stenotic region and surrounding healthy tissue at the treatment site.

In some embodiments disclosed herein, the pressure and volume of balloon inflation are the inputs to the model. For example, a higher pressure needed to achieve a certain angioplasty balloon volume can be used to compute that the blockage stiffness is higher than expected. If imaging data (CT or US, for example) are available then this can serve as further input. This can include final balloon state and/or transient information as the balloon is inflating. This can also include pre-procedural data, such as geometric data obtained from CT imaging data. It can further include geometric data of the lumen after ballooning obtained, for example from angiography or IVUS.

In some embodiments disclosed herein, improved monitoring of the inflation process can be achieved by including a sensor inside the balloon, with examples including an ultrasound, a micro-electromechanical system (MEMS) sensor, a strain gauge sensor, and/or capacitive sensor, and/or by employing a transparent balloon with an internal optical sensor, and/or by including an imageable pattern on the balloon from which its three-dimensional (3D) geometry can be estimated via the imaging.

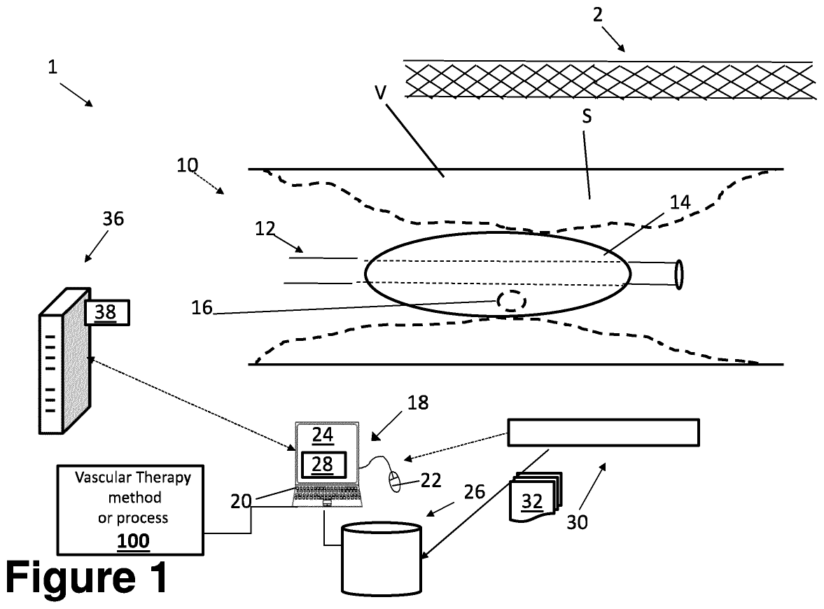

With reference to FIG. 1, an illustrative intravascular therapy (i.e., thrombectomy or atherectomy) apparatus 1 is diagrammatically shown. As shown in FIG. 1, the apparatus 1 includes an angioplasty device 10 for performing an angioplasty procedure at a treatment site of a patient comprising stenotic region S (diagrammatically shown in FIG. 1 with dashed lines) in a blood vessel V of a patient. After the angioplasty procedure is performed, a subsequent intravascular therapy treatment procedure is performed at the same treatment site using the same or (more usually) a different intravascular instrument, such a catheter (not shown) configured to deliver a self-expanding vascular therapy device 2 (e.g., a self-expanding stent, a self-expanding filter, and so forth) into the blood vessel V and to deploy the device 2 at the treatment site. The self-expanding device 2 may, for example, comprise Nitinol. Rather than being self-expanding, it is alternatively contemplated for the vascular therapy device 2 to be expanded at the treatment site using a balloon disposed inside the device 2. In another example, the vascular therapy device 2 can comprise a balloon deployed within the vessel V.

As shown in FIG. 1, the balloon angioplasty device 10 includes a catheter 12 used to perform the balloon angioplasty procedure. The catheter 12 has a balloon 14 disposed at a distal end thereof. The balloon 14 is configured to be inflated via an inflation source (not shown), such as a fluid delivery source. When the balloon 14 is inflated, the outer surface of the balloon 14 pushes against the material of the stenosis S to move the material of the stenosis S radially towards the walls of the vessel V. In some examples, the balloon 14 can be made from a transparent material to allow visualization of the stenosis S and/or the blood vessel V. In some examples, the balloon 14 can be coated with a therapy drug (not shown) for treating at least a portion of the stenosis S. In another example, an outer surface of the balloon 14 can include a pattern of a radiopaque substance (not shown), such that when an image is acquired of the balloon 14 at the treatment site, a 3D shape of the balloon 14 can be recreated from the (two-dimensional (2D)) image. In yet another example, an interior of the balloon 14 can include an optical probe or fiber or sensor (not shown) configured to transmit and receive lights having wavelengths which can be used to perform spectroscopy (i.e., Near infra-red (NIR) spectroscopy) to characterize a type of tissue at the blood vessel V (see, e.g., Khraishah H, Jaffer FA. Intravascular Molecular Imaging: Near-Infrared Fluorescence as a New Frontier. Front Cardiovasc Med. 2020; 7:587100. Published 2020-11-23. doi: 10.3389/fcvm.2020.587100). For fluorescence, fluorophores can be applied or injected locally into the balloon 14 or the patient, which can emit signals when excited by NIR light. For example, the balloon 14 can be coated with fluorophores, and imaged with a NIR spectroscopy probe (not shown) to provide excitation signals that characterize the tissue of the patient.

In some embodiments, a sensor 16 (diagrammatically shown in FIG. 1 as a small circle) can be disposed inside of the balloon 14 to measure data about the angioplasty procedure. The sensor 16 can be, for example, a pressure sensor (to measure a pressure or volume inside of the balloon 14), a flow sensor to measure fluid flow in the balloon 14, a capacitive sensor to track a shape of the balloon 14, an optical sensor such as a camera or fiber probe, an intravascular ultrasound (IVUS) sensor, a MEMS sensor, and so forth. In another example, a pressure sensor (not shown) can be included at an inflation source (not shown) to measure a pressure inside of the balloon 14. The IVUS sensor 16 can be used to determine tissue compression at the treatment site before, during and after ballooning for determining individual tissue properties. The IVUS sensor 16 can either be used in a stationary position thus providing a single 2D data frame, or it can be moved along the axis of the balloon 14 to obtain more detailed 3D data.

FIG. 1 further shows an electronic processing device 18, such as a workstation computer, or more generally a computer. The electronic processing device 18 may also include a server computer or a plurality of server computers, e.g., interconnected to form a server cluster, cloud computing resource, or so forth, to perform more complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth, or a touchscreen configured to receive inputs from a user). In some embodiments, the display device 24 can be a separate component from the workstation 18 or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

FIG. 1 also shows an imaging device 30 configured to acquire images 32 of the angioplasty device 10 during an angioplasty procedure. The imaging device 30 is in communication with the at least one electronic processor 20 of the electronic processing device 18. In particular, in the illustrative examples the imaging device 30 comprises an ultrasound (US) imaging device 30; however, it will be appreciated that any suitable imaging device, such as, X-ray, computed tomography (CT), magnetic resonance imaging (MRI), nuclear imaging, or any other suitable imaging device may be used. In some examples, the imaging device 30 can be a fluoroscopic imaging device (e.g., an X-ray imaging device, C-arm imaging device, a CT scanner, or so forth) and the radiopaque coating pattern (when included) of the outer surface of the balloon 14 is visible under imaging, thereby allowing a position of the angioplasty device 10 relative to the stenosis S to be determined. While the illustrative imaging device 30 is an external device that is placed externally to the patient, it is contemplated for the imaging device to be an intravascular imaging device, for example comprising an ultrasound transducer conveniently located on the catheter 12 (and optionally inside the balloon 14, e.g. IVUS sensor 16). A combination of external and intravascular imaging is further contemplated, with the external and intravascular images being shown side-by-side, fused, or otherwise utilized in combination to provide more detailed information about the angioplasty.

The images 32 can be stored in the non-transitory storage media 26, or in a server computer 36. In addition, the server computer 36 can also store a digital twin 38 (i.e., a simulation model) of the treatment site. The digital twin 38 includes data related to tissue compliance of the stenosis S and surrounding tissue at the treatment site. The digital twin 38 can be used to calculate tissue properties at the treatment site, in some cases using the images 32 or data analyzed by the sensor 16.

The digital twin 38 can be generated from the imaging data 32, which can then be segmented by the server computer 36. A discretized mesh of the segmentation is generated, and mathematical models (not shown) describing material behavior stored in the server computer 36 can be applied to portions of the discretized mesh to generate the digital twin 38. Inputs can be applied to the digital twin 38, and outcome responses by the digital twin 38 to the inputs can be calculated. The outcome responses of the digital twin 38 should match physical observation of the patient, in vivo or in vitro. Parameters of the digital twin 38 can be adapted to change the predicted behavior. In this way parameters such as material properties or geometry can be indirectly determined.

The at least one electronic processor 20 is configured as described above to perform a vascular therapy method or process 100. The non-transitory storage medium 26 stores instructions which are readable and executable by the at least one electronic processor 20 to perform disclosed operations including performing the vascular therapy method or process 100. In some examples, the method 100 may be performed at least in part by cloud processing.

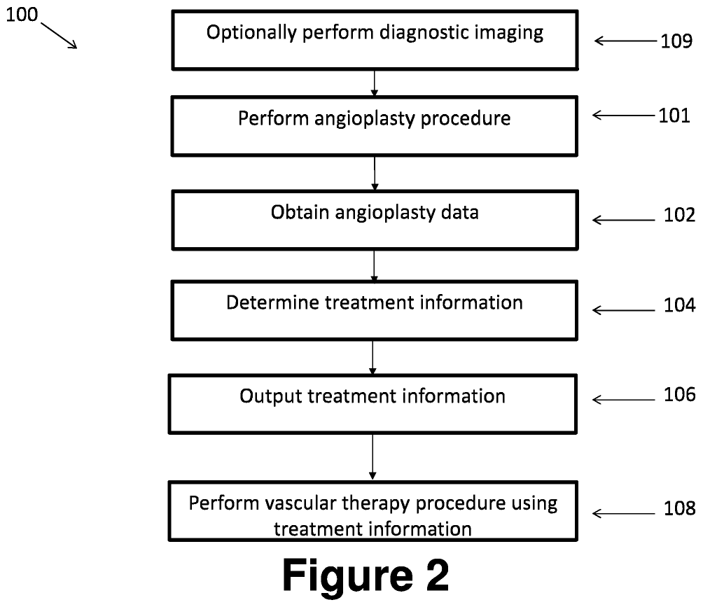

Referring to FIG. 2, and with continuing reference to FIG. 1, an illustrative embodiment of the vascular therapy method 100 is diagrammatically shown as a flowchart. To begin the method 100, at an operation 109 imaging data is optionally obtained using the imaging device 30 and can be performed before or during the angioplasty procedure. This data can serve as input for the digital twin 38.

Next, at an operation 101, the angioplasty procedure is performed. To do so, the angioplasty device 10 is deployed to the treatment site, into the blood vessel V via the therapy device 10, through the stenotic region S, and the balloon 14 is expanded (i.e., inflated) to increase a lumen of the vessel V by applying outward radial pressure on the stenosis S directed towards the walls of the blood vessel V.

At an operation 102, while the angioplasty procedure is performed on the treatment site, data about the angioplasty procedure is obtained by the at least one electronic processor 20. This data can be acquired over time. In one example, the sensor 16 is configured to measure the data about the angioplasty procedure that is obtained by the electronic processor 20. In another example, the imaging device 30 is configured to acquire the data of the treatment site that is obtained by the electronic processor 20. In another example, both the sensor 16 and external imaging device 30 are used concurrently. The obtained data about the angioplasty procedure can more generally include, for example, one or more of an inflation pressure of the balloon 14 (e.g., a maximum pressure and/or maximum expanded volume of the balloon 14), an inflation volume of the balloon 14, and/or imaging data (i.e., the acquired images 32) of the treatment site obtained during the angioplasty procedure. The pressure or volume data of the balloon 14 can be used to determine, for example, a stiffness of tissue at the treatment site. In some examples, the obtaining of data includes measuring the maximum expanded volume of the balloon 14 can employ using the images 32 of the balloon 14 during the balloon angioplasty procedure, or measuring a volume of fluid flowed into the balloon 14 during the angioplasty procedure.

In a variant of the operation 102, transient angioplasty information can be obtained during the process of inflating the balloon 14. For example, if tissue compliance of the stenosis S varies with distance from the inner wall of the blood vessel V, then this may show up as a non-uniform rate of increase in balloon diameter, e.g. portions of the stenosis S that are stiffer will slow the rate of increase of the diameter of the inflating balloon 14. As another example, if the imaging shows the balloon 14 well (for example, if the imaging mode is X-ray and the balloon 14 is coated with a radiopaque film or equipped with a radiopaque pattern) but does not provide a well-defined edge for the clot C, then initial contact of the inflating balloon with the clot S may be detected as an abrupt decrease or brief stoppage of the rate of increase in the diameter of the inflating balloon. Hence, the size of the lumen of the stenotic vessel V can be at the beginning of the angioplasty can be derived from the balloon diameter at the time of that abrupt decrease or stoppage. These are merely illustrative examples.

At an operation 104, treatment information for a subsequent vascular therapy treatment of the treatment site can be determined by the electronic processor 20 by analyzing the data about the angioplasty procedure obtained in the obtaining operation 102. In some embodiments, the analyzing of the data about the angioplasty procedure to determine the treatment information includes analyzing the maximum pressure and/or maximum expanded volume of the balloon 14 during the angioplasty procedure to determine the treatment information comprising a tissue stiffness at the treatment site.

In some embodiments, the treatment information determination operation 104 can include retrieving the digital twin 38 from the server computer 36, and using the retrieved digital twin 38 to analyze the obtained data. To do so, the obtained is input to the digital twin 38, the electronic processor 20 analyzes the digital twin 38 to determine the treatment information.

In some examples, the determined treatment information includes a selection of the vascular therapy device 2 to be used in the subsequent vascular therapy treatment procedure. The selection of the vascular therapy device 2 can include, for example, a selection of a stiffness of the vascular therapy device 2 determined based at least in part on the determined compliance of the tissue of the stenosis S, and/or a diameter of the self-expanding vascular therapy device 2 determined based at least in part on the maximum diameter of the inflated balloon 14 during the angioplasty procedure (as that maximum diameter is indicative of the lumen diameter provided by the remodeling of the stenosis S produced by the angioplasty). The vascular therapy device 2 can be larger than the diameter of the vessel lumen, in order to apply a radial force against the stenosis S. In a particular example, when the obtained data includes an inflation pressure or volume of the balloon 14, the determined treatment information includes at least one tissue property of treatment site, such as a compliance of healthy tissue at the treatment site, a blockage stiffness of the stenosis C, and so forth.

At an operation 106, the treatment information is output to a medical professional performing the angioplasty procedure, such as on the display device 24 of the electronic processing device 18.

At an operation 108, the subsequent vascular therapy treatment procedure of the treatment site is performed in accordance with the determined treatment information. For example, the vascular therapy treatment procedure can include deploying the self-expanding vascular therapy device 2 with the determined information (e.g., a determined diameter and/or stiffness, or length of the self-expanding vascular therapy device 2).

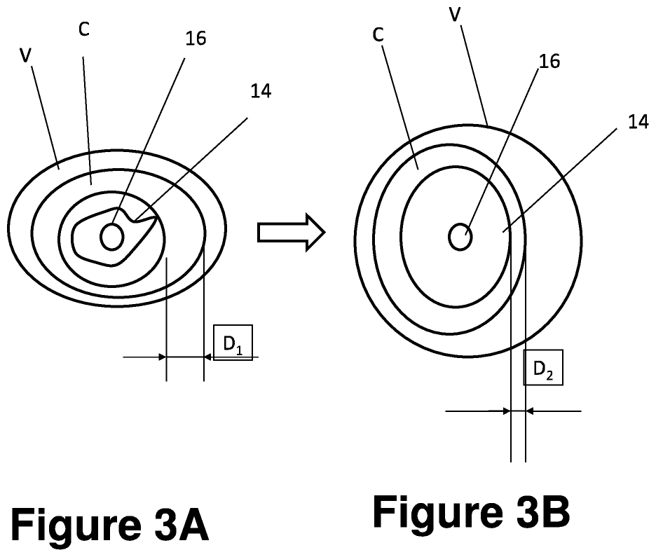

FIGS. 3A and 3B show an example of the angioplasty method. FIG. 3A shows the balloon 14 in an uninflated state in the blood vessel V next to the stenosis S, for example caused by a plaque layer. The IVUS sensor 16 is also shown. The plaque layer has a first diameter D1. FIG. 3B shows the balloon 14 in an inflated state to compress the plaque layer against the walls of the blood vessel V. The plaque layer has a second diameter D2 that is smaller than the first diameter due to the compression of the clot by the inflated balloon 14. Additional information can be extracted from the change in diameter and thicknesses of tissue and plaque layers in combination with for example balloon shape and pressure.

As previously noted, the balloon angioplasty device 10 may comprise the catheter 12, the expandable balloon 14 disposed at a distal end of the catheter, and the sensor 16 disposed inside the balloon and configured to measure data about an angioplasty procedure performed using the catheter. The sensor 16 is useful for obtaining angioplasty data in the obtaining operation 102. More generally, however, the sensor 16 (which may, for example, comprise a pressure sensor to measure a pressure inside of the balloon 14, a flow sensor to measure fluid flow in the balloon 14, a capacitive sensor to track a shape of the balloon 14, an optical sensor, an IVUS sensor, or so forth) can be useful apart from the method 100. For example, monitoring pressure and/or fluid flow inside the balloon 14 during the angioplasty procedure, and especially during balloon inflation or over a time period during which a maximum pressure is intended to be held constant, can be useful to detect a failure (e.g. leak in) the balloon 14. In another embodiment, the sensor 16 is an optical sensor, the balloon 14 is transparent for the wavelength or wavelength range at which the optical sensor operates, and the balloon 14 further includes an imageable pattern on the balloon from which its three-dimensional (3D) geometry can be estimated via the imaging by the (in this embodiment optical) sensor 16. For example, the imageable pattern can be a grid made of a coating of material that is translucent or absorbing for the operating wavelength or wavelength range of the optical sensor 16. If the optical sensor 16 is placed at the center of the balloon 16, then optical monitoring of the imageable pattern using the optical sensor 16 enables determination of whether the balloon expands isotopically from its center, and more generally can provide a detailed 3D surface rendering of the expanded balloon 14.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A vascular therapy apparatus, comprising:
an electronic processing device comprising at least one electronic processor programmed to:
while an angioplasty procedure is performed on a treatment site in a vessel (V) of a patient, obtain data about the angioplasty procedure;
determine treatment information for a subsequent vascular therapy treatment of the treatment site by analyzing the data about the angioplasty procedure; and
output the treatment information.

2. The vascular therapy apparatus of claim 1, wherein the electronic processor is programmed to determine the treatment information by operations including:
retrieving a digital twin of the treatment site from a database; and
determining the treatment information by analyzing the data about the angioplasty procedure using the retrieved digital twin.

3. The vascular therapy apparatus of claim 2, wherein the digital twin is configured to determine data related to tissue compliance of a stenosis and surrounding tissue at the treatment site.

4. The vascular therapy apparatus of claim 1, wherein:
the obtained data about the angioplasty procedure includes one or more of inflation pressure of an associated balloon used in the angioplasty procedure, inflation volume of the associated balloon used in the angioplasty procedure, and/or imaging data of the treatment site obtained before, during and/or after the angioplasty procedure; and
the determined treatment information includes a selection of an associated vascular therapy device to be used in the subsequent vascular therapy treatment.

5. The vascular therapy apparatus of claim 4, wherein the selection of the associated vascular therapy device includes selection of at least one of a stiffness of the associated self-expanding vascular therapy device to be used in the subsequent vascular therapy treatment and a diameter of the associated self-expanding vascular therapy device to be used in the subsequent vascular therapy treatment, and a length of the associated self-expanding vascular therapy device to be used in the subsequent vascular therapy treatment.

6. The vascular therapy apparatus of claim 1, wherein:
the obtained data about the angioplasty procedure includes at least one of inflation pressure and inflation volume of an associated balloon used in the angioplasty procedure; and
the determined treatment information includes at least one tissue property of treatment site.

7. The vascular therapy apparatus of claim 2, wherein the obtained data comprises inflation volume of an associated balloon used in the angioplasty procedure, and the at least one electronic processor is programmed to:
input inflation volume to the digital twin;
analyze the digital twin; and
output the treatment information comprising a blockage stiffness of the stenosis (S).

8. The vascular therapy apparatus of claim 1, wherein the at least one electronic processor is programmed to:
output the treatment information on a display device of the electronic processing device.

9. The vascular therapy apparatus of claim 1, further comprising:
a catheter having a balloon disposed at a distal end thereof, the catheter being used to perform the angioplasty procedure.

10. The vascular therapy apparatus of claim 9, further comprising:
a sensor disposed inside of the balloon, the sensor configured to measure the data about the angioplasty procedure that is obtained by the electronic processor.

11. The vascular therapy apparatus of claim 10, wherein the sensor comprises one of a pressure sensor, an ultrasound sensor, a capacitive sensor, or an optical sensor.

12. The vascular therapy apparatus of claim 9, further comprising:
a vascular therapy device used to perform the subsequent vascular therapy treatment, the self-expanding vascular therapy device being deployed using the treatment information.

13. The vascular therapy device (1) of claim 8, further comprising:
an imaging device configured to acquire the data of the treatment site that is obtained by the electronic processor.

14. The vascular therapy device of claim 13, wherein the imaging device comprises one of a computed tomography (CT) imaging device or an ultrasound (US) imaging device or an X-ray imaging device.

15. A vascular therapy method comprising:
performing an angioplasty procedure by expanding a balloon at a treatment site in a vessel (V) of a patient;
while performing the angioplasty procedure, obtaining data about the angioplasty procedure;
analyzing the data about the angioplasty procedure using an electronic processor to determine treatment information for a subsequent vascular therapy treatment of the treatment site; and
performing the subsequent vascular therapy treatment of the treatment site in accordance with the determined treatment information.

16. The vascular therapy method of claim 15, wherein the obtaining of data includes measuring a maximum pressure and/or maximum expanded volume of the balloon during the angioplasty procedure.

17. The vascular therapy method of claim 16, wherein the obtaining of data includes measuring the expanded volume of the balloon using imaging of the balloon during the balloon angioplasty procedure or measuring a volume of fluid flowed into the balloon during the angioplasty procedure.

18. The vascular therapy method of claim 16, wherein the analyzing of the data about the angioplasty procedure to determine the treatment information includes analyzing the pressure and/or expanded volume of the balloon during the angioplasty procedure to determine the treatment information comprising a tissue stiffness at the treatment site.

19. The vascular therapy method of claim 16, wherein:
the analyzing of the data about the angioplasty procedure to determine the treatment information includes analyzing the pressure and/or expanded volume of the balloon during the angioplasty procedure to determine the treatment information including a diameter and/or stiffness of an intravascular instrument for use in the subsequent vascular therapy treatment; and
the performing of the subsequent vascular therapy treatment of the treatment site in accordance with the determined treatment information includes performing of the subsequent vascular therapy treatment using an interventional instrument with the determined diameter and/or stiffness.

20. A balloon angioplasty device, comprising:
a catheter;
an expandable balloon disposed at a distal end of the catheter; and
a sensor disposed inside the balloon and configured to measure data about an angioplasty procedure performed using the catheter.
